(19) United States
(12) Patent Application Publication
(10) Pub. No.: US 2014/0041667 A1
(43) Pub. Date: Feb. 13, 2014

(54) Intra Uterine Device For An Animal And A System For Loading The Intra Uterine Device Into A Delivery Gun

(71) Applicant: Scott Cammack, Sturgis, SD (US)

(72) Inventor: Scott Cammack, Sturgis, SD (US)

(21) Appl. No.: 13/928,277

(22) Filed: Jun. 26, 2013

Related U.S. Application Data
(60) Provisional application No. 61/680,536, filed on Aug. 7, 2012.

(51) Int. Cl. A61F 6/18; A61K 33/34; A61F 6/14

(52) U.S. Cl. CPC A61F 6/18; A61F 6/144; A61K 33/34
USPC 128/833

(57) ABSTRACT

A system for delivering an IUD into an animal, including: (i) an IUD for a an animal, comprising: (a) a main body; and (b) a pair of bendable arms extending away from the main body, wherein the ends of the bendable arms fold together in a direction away from the main body; (ii) a delivery gun for inserting the IUD into an animal, wherein the delivery gun comprises a tubular barrel and a plunger moveable within the tubular barrel; and (iii) a loading gun for inserting the IUD into the tubular barrel of the delivery gun, wherein the loading gun is configured to insert the IUD into the delivery gun with the pair of bendable arms being folded together forward of the main body of the IUD.

### RELATED APPLICATION

The present application claims priority to U.S. Provisional Patent Application Ser. No. 61/680,536, filed Aug. 7, 2012, of same title.

### TECHNICAL FIELD

The present invention relates to Intra Uterine Devices (IUDs) for animals in general and cattle in particular.

### BACKGROUND OF THE INVENTION

A benefit of using bovine IUDs are that they increase cow weight gain which is desired by ranchers. Unfortunately, inserting an IUD into a cow is a tricky and time consuming process. What is instead desired is a quick and easy system to insert IUDs into a herd of cattle in a relatively short period of time. An additional benefit of designing and building a rapid IUD delivery system for cattle is that such IUS may also be optionally used as a standard contraceptive for feral horses.

### SUMMARY OF THE INVENTION

The present invention provides a non-human IUD that is ideally suited for bovine and ruminant use. In addition, however, it may also be used with other animals as desired.

The present invention also provides a delivery gun for the IUD placement in the animal, and a loading gun for inserting the IUD into the delivery gun. An advantage of the loading and delivery guns are that IUDs can be quickly loaded and then delivered into a herd of cattle in a relatively short period of time. In addition, the system is safe, fast and easy to use.

In preferred aspects, the present invention provides a system for delivering an IUD into an animal, including: (i) an IUD for a an animal, comprising: (a) a main body; and (b) a pair of bendable arms extending away from the main body, wherein the ends of the bendable arms fold together in a forward direction away from the main body; (ii) a delivery gun for inserting the IUD into an animal, wherein the delivery gun comprises a tubular barrel and a plunger moveable within the tubular barrel; and (iii) a loading gun for inserting the IUD into the tubular barrel of the delivery gun, wherein the loading gun is configured to insert the IUD into the delivery gun with the pair of bendable arms being folded together forward of the main body of the IUD.

In use, a veterinarian first places the main of the IUD in the barrel of the delivery gun with the bendable arms extending outwardly. Next, the veterinarian uses a loading gun to push the IUD down fully into the delivery gun (such that the bendable arms fold together pointing forward in the distal direction towards the delivery end of the barrel). The delivery gun is then used to place the IUD in the animal's uterus.

### BRIEF DESCRIPTION OF THE DRAWINGS

FIG. 1 is an illustration of the IUD sitting next to the distal end of the delivery gun.

FIG. 2 is an illustration of the IUD after it has been hand placed into the distal end of the delivery gun.

FIG. 3 is an illustration of the distal end of the delivery gun about to be placed into the loading gun.

FIG. 4 is an illustration of the distal end of the delivery gun after it has been placed into the loading gun.

FIG. 5 is an illustration of the loading plunger in the loading gun advancing to a first position to start to push the IUD down into the barrel of the delivery gun.

FIG. 6 is a an illustration of the loading plunger in the loading gun advancing to a second position pushing the IUD down into the barrel of the delivery gun.

FIG. 7 is an illustration of the loading plunger after it has fully pushed the IUD down into the distal end of the delivery gun.

FIG. 8 is an illustration after the loading plunger has been retracted, prior to removing the delivery gun from the loading gun.

FIG. 9 is a perspective view of the delivery gun.

### DETAILED DESCRIPTION OF THE DRAWINGS

The present invention provides an IUD (intra-uterine device) for a an animal in general, and for cattle and horses in particular. The present IUD may be used (in the case of cattle) to promote weight gain, or (in the case of horses) for contraception.

Studies have shown good cattle weight gain with IUD usage. Spaying (the current technique) has a mortality rate of 1 to 3 cows per 1000 head. It is also hard on the animals physically and leads to less weight gain than for non-spayed, non-IUD cows. The present invention overcomes these problems.

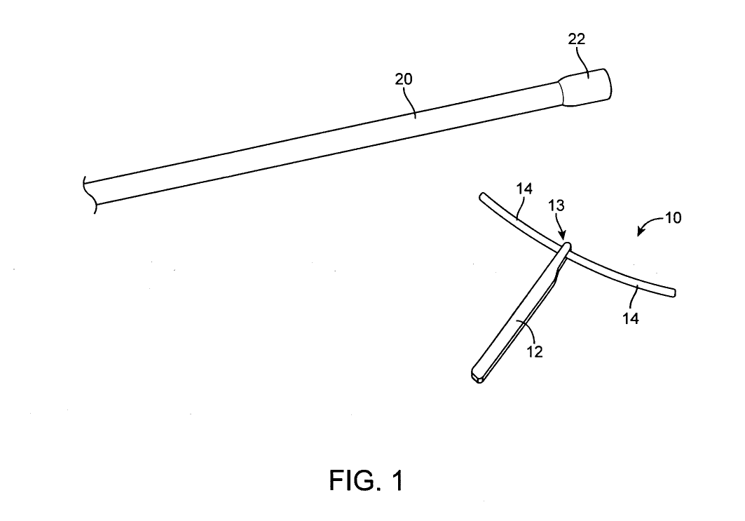

In preferred aspects, the present IUD 10 (FIG. 1), comprises a main body 12; and a pair of bendable arms 14 extending away from the main body. Optionally, the pair of bendable arms are opposing portions of a single bendable member 14 that is received through a hole 13 passing through the end of main body 12.

Preferably as well, main body 12 has an exterior surface made of copper, and bendable member 14 is made of monofilament nylon. Optionally, the main body 12 further comprises a plastic inner core under the copper surface. The copper surface may optionally be etched to further increase its surface area. Optionally, as well, a string (not shown) may be attached to main body to permit retrieval of the IUD after insertion into the animal.

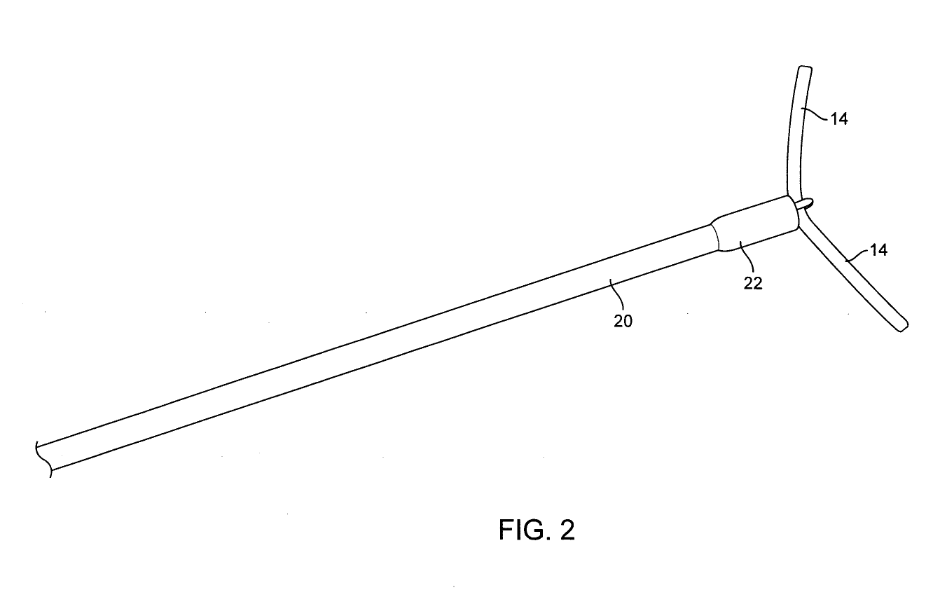

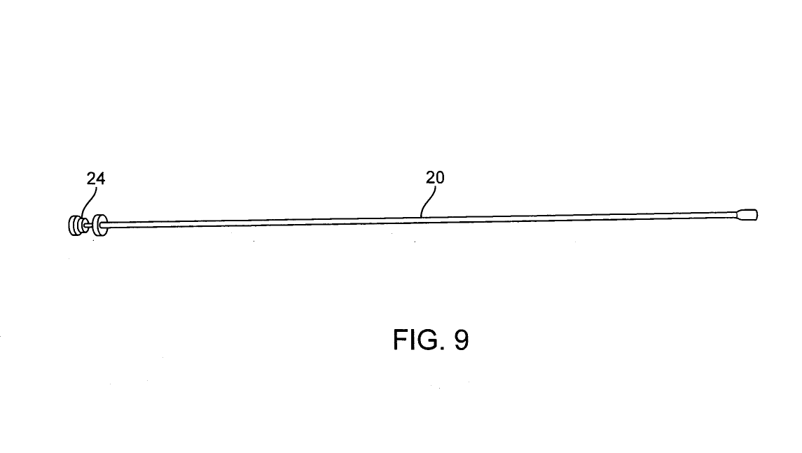

Turning next to FIG. 2, IUD 10 is hand loaded into the barrel 20 of a delivery gun. As can be seen, bendable arms 14 project outwardly. As can also be seen, the barrel of the delivery gun 20 has a rounded and bulbous distal end 22 into which the IUD is loaded. In preferred embodiments, delivery gun 20 is a modified artificial insemination gun. The delivery gun 20 comprises a tubular barrel with a plunger 24 (FIG. 9) moveable within the tubular barrel.

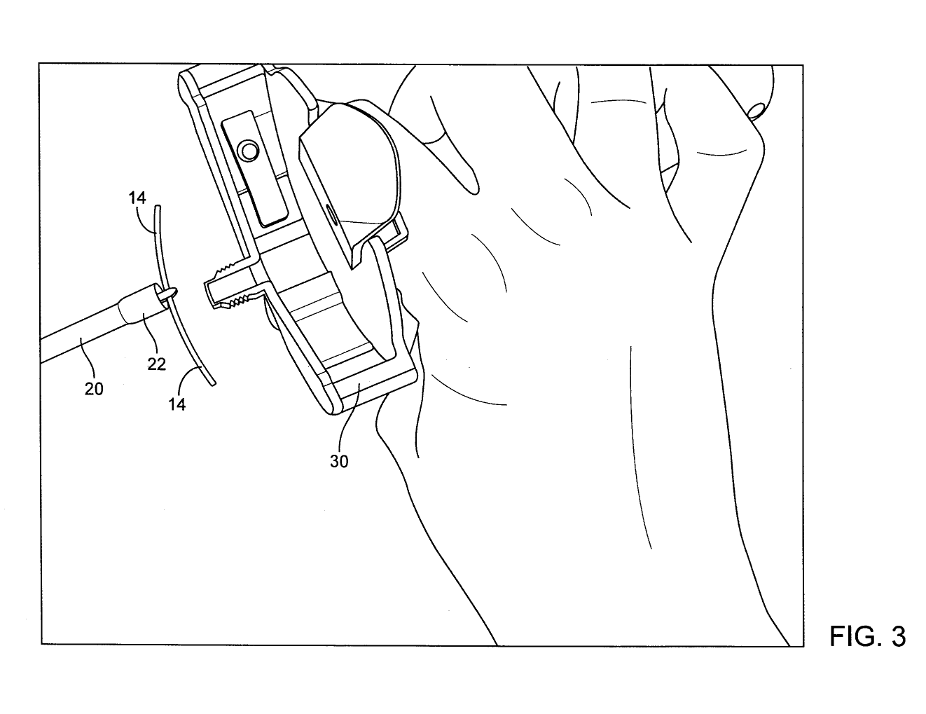

Turning next to FIG. 3, the distal end of the delivery gun 20 is about to be placed into a loading gun 30. Loading gun 30 is used to fully insert IUD 10 into the tubular barrel of delivery gun 20 with the pair of bendable arms 14 being folded together forward of the main body 12 of IUD 10. In preferred embodiments, loading gun 30 is a modified hormone implant gun.

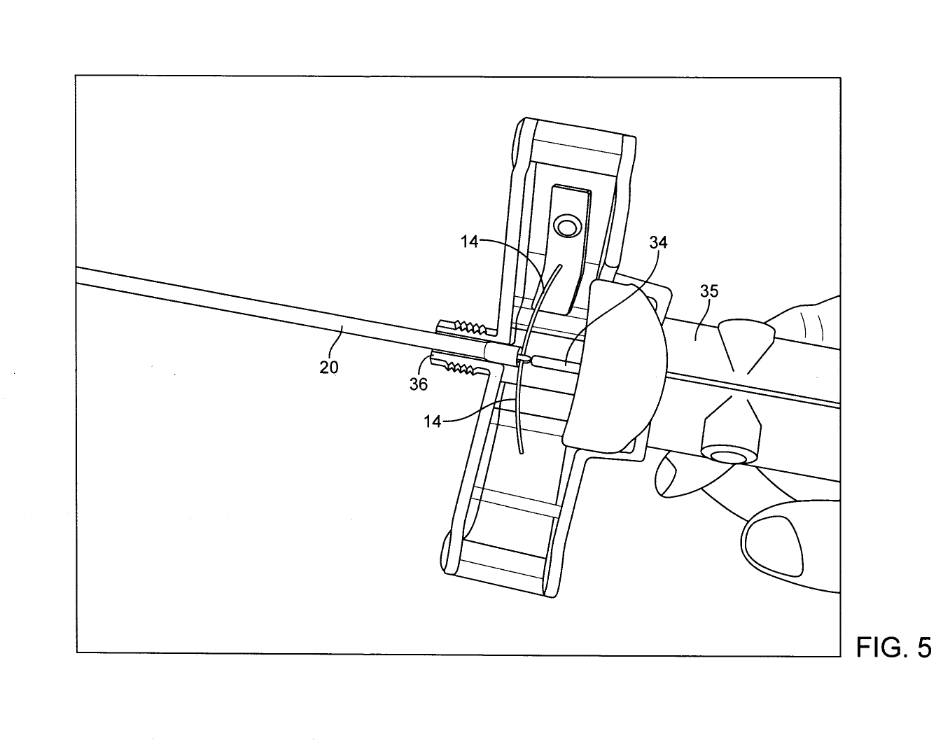

Loading gun 30 comprises: a frame 32 for receiving the distal end of the delivery gun therein; a loading plunger 34 aligned with frame 32 for pushing the IUD down into the distal end 22 of the tubular barrel of delivery gun 20; and a handle 35 (FIG. 5). Preferably, the handle further includes a trigger mechanism for advancing loading plunger 34.

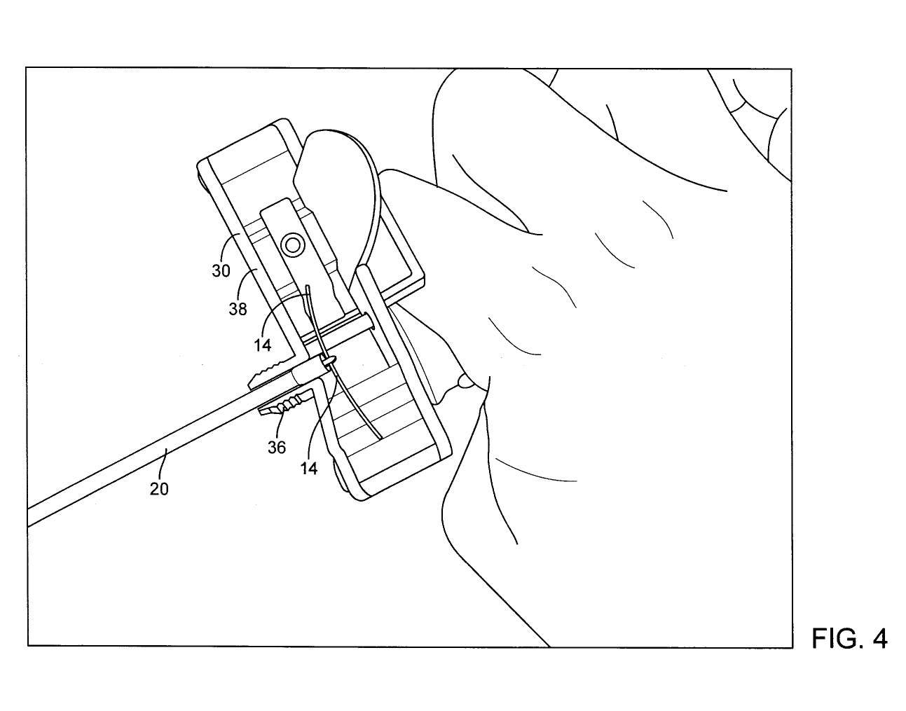

Next, as shown in FIG. 4, the distal end 22 of delivery gun 20 is received into a receptacle 36. Receptacle 36 is dimensioned to receive the tubular barrel of the delivery gun therein, and to hold the barrel straight. Loading gun 30 also comprises a flat panel 38 adjacent to receptacle 36, but spaced apart from the body of the handle such that the pair of bendable arms 14 on the IUD extend outwards (as shown). Specifically, receptacle 36 preferably surrounds a portion of the circumference of the tubular barrel of delivery gun 20, as shown.

Next, as seen in FIG. 5, the veterinarian pulls the trigger of loading gun 30, causing loading plunger 34 to advance into contact with the distal end of main body 12.

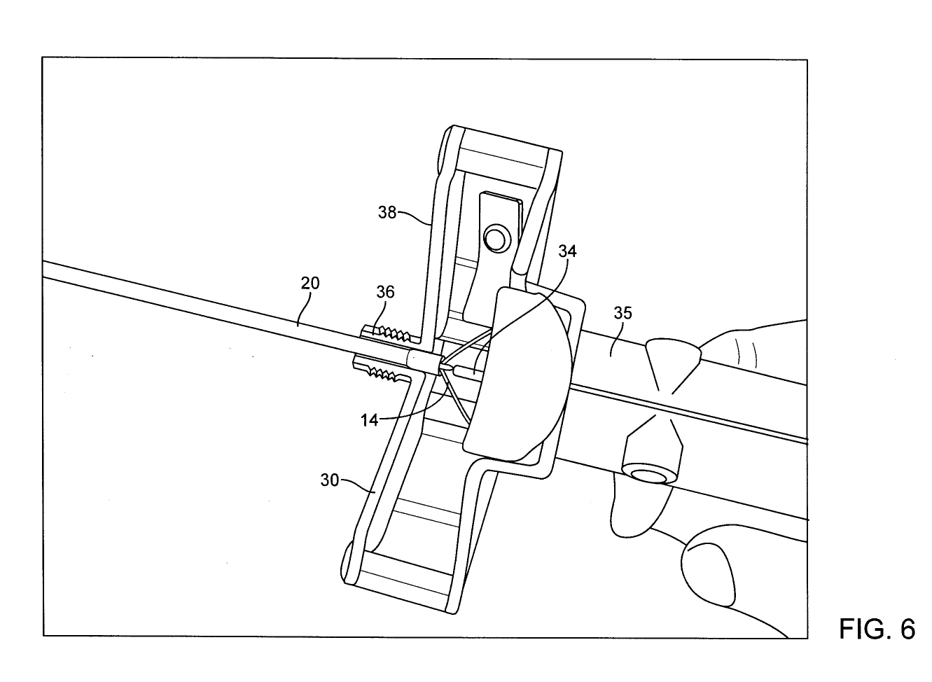

Next, as seen in FIG. 6, loading plunger 34 is further advanced causing the ends of bendable arms 14 to fold together (in a forward-pointing direction away from main body 12).

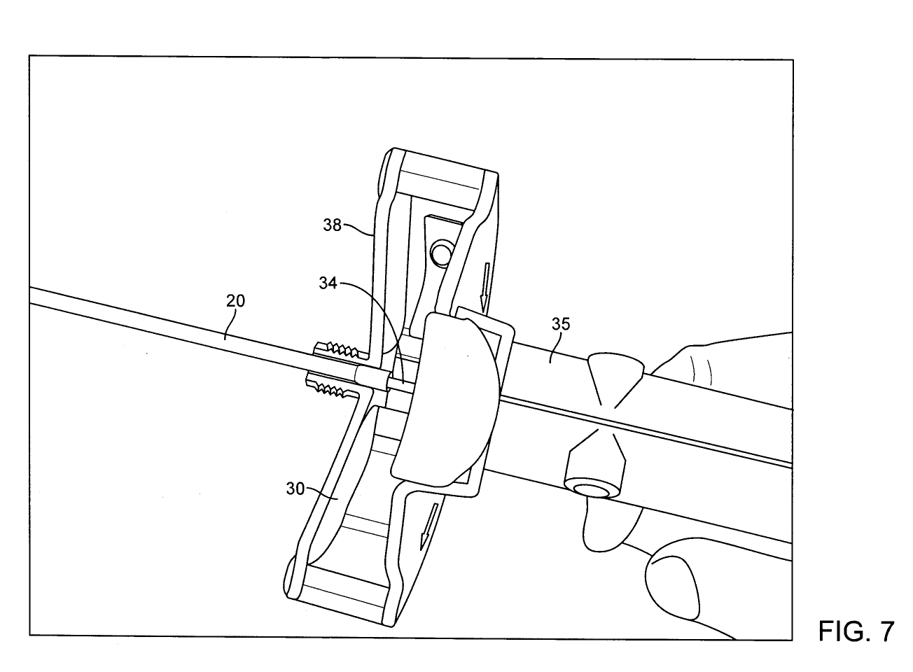

Next, as seen in FIG. 7, loading plunger 34 will push IUD 10 down fully within the distal end of the barrel of delivery gun 20. At this time, the pair of bendable arms 14 will be folded together at the distal end of the barrel of delivery gun 20. (Thus, when the IUD is placed into the uterus of the animal, it will be pushed out of the distal end of the barrel of delivery gun 20 with bendable arms 14 coming out of the delivery gun first followed by the main body of the IUD).

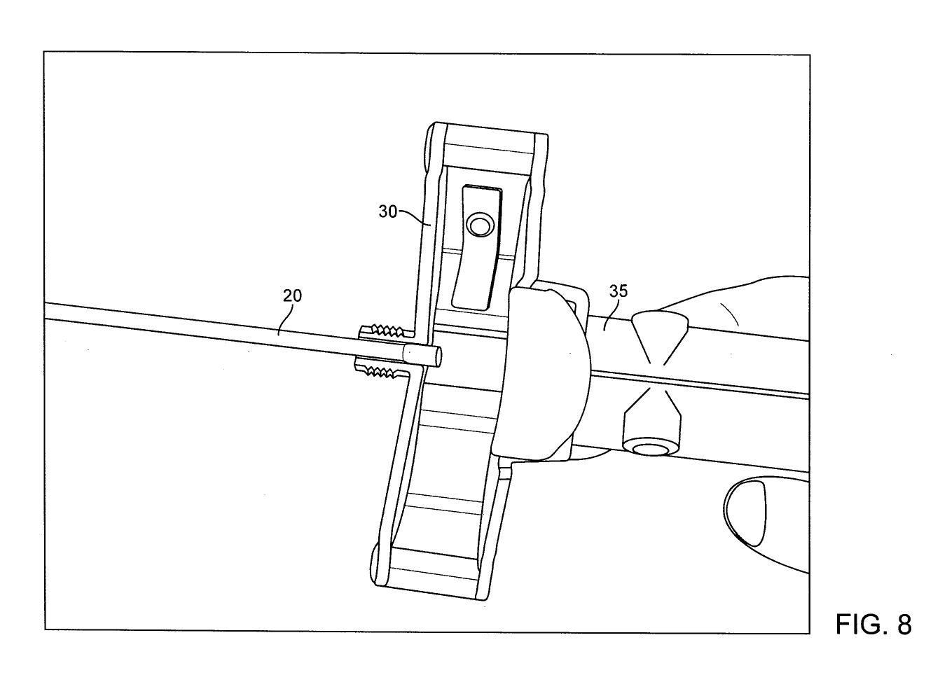

As seen in FIG. 8, loading plunger 34 is retracted back into the body of loading gun 30, and delivery gun 20 is then removed from loading gun 30. As seen in FIG. 9, the distal end (i.e.: tubular barrel) of delivery gun 20 can then be inserted into the uterus of the animal. A delivery plunger 34 in the delivery gun is then advanced, thereby pushing IUD 10 out of the delivery gun, and into the uterus of the animal.

What is claimed is:

1. An IUD for a an animal, comprising:
a main body; and
a pair of bendable arms extending away from the main body, wherein the ends of the bendable arms fold together in a direction away from the main body.

2. The IUD of claim 1, wherein the pair of bendable arms are opposing portions of a single bendable member received through a hole passing through the main body.

3. The IUD of claim 2, wherein the main body has an exterior surface made of copper, and wherein the bendable member is made of monofilament nylon.

4. The IUD of claim 3, wherein the main body further comprises a plastic inner core under the copper surface.

5. The IUD of claim 2, wherein the copper is etched to increase its surface area.

6. A system for delivering an IUD into an animal, comprising:
the IUD of claim 1;
a delivery gun for inserting the IUD into an animal, wherein the delivery gun comprises a tubular barrel and a plunger moveable within the tubular barrel; and
a loading gun for inserting the IUD into the tubular barrel of the delivery gun, wherein the loading gun is configured to insert the IUD into the delivery gun with the pair of bendable arms being folded together forward of the main body of the IUD.

7. The system of claim 6, wherein the delivery gun has a rounded and bulbous distal end into which the IUD is loaded.

8. The system of claim 6, wherein the delivery gun is a modified artificial insemination gun.

9. The system of claim 6, wherein the loading gun is a modified hormone implant gun.

10. The system of claim 6, wherein the loading gun comprises:
a frame for receiving the distal end of the delivery gun therein;
a loading plunger aligned with the frame for pushing the IUD down into the distal end of the tubular barrel of the delivery gun; and
a handle.

11. The system of claim 10, wherein the handle further comprises:
a trigger mechanism for advancing the loading plunger.

12. The system of claim 10, wherein the frame comprises:
a receptacle dimensioned to receive the tubular barrel of the delivery gun therein; and
a flat panel adjacent to the receptacle and spaced apart from the handle such that the pair of bendable arms on the IUD can extend outwards prior to the loading plunger pushing the IUD down into the tubular barrel of the delivery gun, thereby folding the pair of bendable arms together.

13. The system of claim 12, wherein the receptacle surrounds a portion of the circumference of the tubular barrel of the delivery gun.

14. A method of delivering an IUD into an animal, comprising:
placing an IUD into tubular barrel of a delivery gun;
placing the tubular barrel of the delivery gun into a loading gun;
advancing a loading plunger in the loading gun, thereby pushing the IUD down into the distal end of the tubular barrel such that a pair of bendable arms on the IUD fold together in a direction away from a main body of the IUD;
removing the delivery gun from the loading gun;
inserting the delivery gun into the uterus of an animal;
advancing a delivery plunger in the delivery gun, thereby pushing the IUD out of the delivery gun, and into the uterus of the animal.

15. The method of claim 14, wherein placing the tubular barrel of the delivery gun into the loading gun comprises:
positioning the tubular barrel of the delivery gun into a receptacle on the frame of the loading gun with the main body of the IUD aligned with the loading plunger in the loading gun, and with the bendable arms of the IUD projecting outwardly into a space in the frame of the loading gun.
